(19) United States
(12) Patent Application Publication
(10) Pub. No.: US 2026/0076580 A1
(43) Pub. Date: Mar. 19, 2026

(54) PHOTOACOUSTIC DEVICES CONFIGURED FOR BLOOD PRESSURE ESTIMATION

(71) Applicant: QUALCOMM Incorporated, San Diego, CA (US)

(72) Inventors: Sumit AGRAWAL, Sunnyvale, CA (US); Hrishikesh Vijaykumar PANCHAWAGH, Cupertino, CA (US); Emily Kathryn BROOKS, Amherst, NY (US); Kostadin Dimitrov DJORDJEV, Los Gatos, CA (US); John Keith SCHNEIDER, Williamsville, NY (US)

(21) Appl. No.: 18/885,349

(22) Filed: Sep. 13, 2024

(51) Int. Cl. A61B 5/021; A61B 5/00; A61B 5/026

(52) U.S. Cl. CPC A61B 5/02125; A61B 5/0095; A61B 5/026; A61B 5/681

(57) ABSTRACT

Some disclosed examples involve controlling, by a control system of an apparatus, a light source system of the apparatus to provide light to a target object on an outer surface of the apparatus and receiving, by the control system, ultrasonic receiver signals from each of a plurality of M ultrasonic receiver elements in a single array, the ultrasonic receiver signals corresponding to ultrasonic waves generated by the target object responsive to the light. Some disclosed examples involve detecting, by the control system, an artery within the target object based on the ultrasonic receiver signals and estimating, by the control system, a blood pressure within the artery based on the ultrasonic receiver signals.

### TECHNICAL FIELD

This disclosure relates to photoacoustic devices and more specifically to estimating blood pressure based, at least in part, in data obtained via photoacoustic devices.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive and ambulatory monitoring. Some such devices are, or include, photoacoustic devices. Although some photoacoustic devices and systems have previously been deployed, improved photoacoustic devices and systems would be desirable.

### SUMMARY

The systems, methods and devices of this disclosure each have several aspects, no single one of which is solely responsible for the desirable attributes disclosed herein.

One innovative aspect of the subject matter described in this disclosure can be implemented in an apparatus. The apparatus may include a light source system configured to provide light to a target object on an outer surface of the apparatus, an ultrasonic receiver system configured to receive ultrasonic waves generated by the target object responsive to the light from the light source system and a control system. The receiver system may be, or may include, a single array of M ultrasonic receiver elements, where M is an integer of 2 or more. The light source system may include N light sources, where N is an integer of 1 or more. In some implementations, a mobile device (such as a wearable device, a hand-held device, etc.) may be, or may include, at least part of the apparatus.

The control system may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system may be configured to control the light source system to provide light to the target object. The control system may be configured to receive ultrasonic receiver signals from each of a plurality of the M ultrasonic receiver elements in the single array. The ultrasonic receiver signals may correspond to the ultrasonic waves generated by the target object responsive to the light. The control system may be configured to detect an artery within the target object based, at least in part, on the ultrasonic receiver signals. The control system may be configured to estimate a blood pressure within the artery based, at least in part, on the ultrasonic receiver signals.

In some implementations, the apparatus may be configured to be worn on a human wrist. However, in other implementations, the apparatus may be configured to be a hand-held device. According to some examples, the artery may be a radial artery.

In some examples, the control system may be configured to apply a receiver-side beamforming process to the ultrasonic receiver signals, to produce a beamformed ultrasonic receiver image. In some such examples, the control system may be further configured to estimate a change in cross-sectional area of the artery based at least in part on the beamformed ultrasonic receiver image. Estimating the blood pressure may be based in part on the change in the cross-sectional area of the artery.

According to some examples, the control system may be further configured to estimate a change in a blood flow rate within the artery based on the ultrasonic receiver signals. In some such examples, estimating the blood pressure may be based in part on the change in the blood flow rate within the artery and the change in cross-sectional area of the artery.

In some examples, wherein estimating the blood pressure may be based in part on a derivative of the blood flow rate (Q) within the artery with respect to the cross-sectional area (A) of the artery (dQ/dA). In some such examples, the control system may be configured to estimate a pulse wave velocity based on dQ/dA.

According to some examples, the control system may be configured to estimate the change in the blood flow rate within the artery based at least in part on speckle decorrelation-based velocimetry imaging, a continuous wave photoacoustic doppler method, a structured-illumination photoacoustic doppler method, cross-correlation-based flow imaging in a time domain or cross-correlation-based flow imaging in a spatial domain.

In some implementations, the apparatus may include a magnetic sensor system. In some such examples, the control system may be further configured to estimate a change in a blood flow rate within the artery based on magnetic sensor signals from the magnetic sensor system. Estimating the blood pressure may be based in part on the change in the blood flow rate within the artery and the change in cross-sectional area of the artery.

According to some examples, the apparatus may include an optical sensor system. In some such examples, the control system may be further configured to estimate a change in a blood flow rate within the artery based on optical sensor signals from the optical sensor system. Estimating the blood pressure may be based in part on the change in the blood flow rate within the artery and the change in cross-sectional area of the artery.

In some examples, the control system may be configured to receive ultrasonic receiver signals from the ultrasonic receiver system at a frame rate in a range from 1 KHz to 3 KHz. According to some examples, the ultrasonic receiver signals include frequencies in a range from 10 MHz to 25 MHz. In some examples, the single array of M ultrasonic receiver elements may be linearly arranged. According to some examples, the N light sources include one, two, or more vertical-cavity surface-emitting lasers (VCSELs).

Other innovative aspects of the subject matter described in this disclosure can be implemented in one or more methods. Some methods may involve controlling, by a control system of an apparatus, a light source system of the apparatus to provide light to a target object on an outer surface of the apparatus. Some methods may involve receiving, by the control system, ultrasonic receiver signals from each of a plurality of M ultrasonic receiver elements in a single array. The ultrasonic receiver signals may correspond to ultrasonic waves generated by the target object responsive to the light. Some methods may involve detecting, by the control system, an artery within the target object based on the ultrasonic receiver signals. Some methods may involve estimating, by the control system, a blood pressure within the artery based on the ultrasonic receiver signals.

Some methods may involve estimating a change in cross-sectional area of the artery based at least in part the ultrasonic receiver signals. In some examples, estimating the blood pressure may be based in part on the change in the cross-sectional area of the artery. Some methods may involve estimating a change in a blood flow rate within the artery based on the ultrasonic receiver signals. In some examples, estimating the blood pressure may be based in part on the change in the blood flow rate within the artery.

Some or all of the methods described herein may be performed by one or more devices according to instructions (e.g., software) stored on non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in one or more non-transitory media having software stored thereon. The software may include instructions for controlling one or more devices to perform one or more disclosed methods. Some methods may involve controlling, by a control system of an apparatus, a light source system of the apparatus to provide light to a target object on an outer surface of the apparatus. Some methods may involve receiving, by the control system, ultrasonic receiver signals from each of a plurality of M ultrasonic receiver elements in a single array. The ultrasonic receiver signals may correspond to ultrasonic waves generated by the target object responsive to the light. Some methods may involve detecting, by the control system, an artery within the target object based on the ultrasonic receiver signals. Some methods may involve estimating, by the control system, a blood pressure within the artery based on the ultrasonic receiver signals.

Some methods may involve estimating a change in cross-sectional area of the artery based at least in part the ultrasonic receiver signals. In some examples, estimating the blood pressure may be based in part on the change in the cross-sectional area of the artery. Some methods may involve estimating a change in a blood flow rate within the artery based on the ultrasonic receiver signals. In some examples, estimating the blood pressure may be based in part on the change in the blood flow rate within the artery.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1 is a block diagram that shows example components of an apparatus according to some disclosed implementations.

FIG. 2 shows an example of a previously-deployed PAPG-based device for BP estimation.

FIG. 3 shows an example of a PAPG-based device for BP estimation according to the present disclosure.

FIG. 4A shows an example of an ultrasonic receiver system and a light source system integrated into a single photoacoustic (PA) sensor.

FIG. 4B shows an example of the light source system of FIG. 4A.

FIG. 4C shows an example of the ultrasonic receiver system of FIG. 4A.

FIG. 5 shows another example of an ultrasonic receiver system and a light source system integrated into a single photoacoustic (PA) sensor.

FIG. 6 shows an example of an apparatus that is configured to perform a receiver-side beamforming process.

FIG. 7 is a flow diagram that shows examples of some disclosed operations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, autonomous or semi-autonomous vehicles, drones, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

Non-invasive health monitoring devices, such as photoacoustic plethysmography (PAPG)-capable devices, have various potential advantages over more invasive health monitoring devices such as cuff-based or catheter-based blood pressure (BP) measurement devices. However, it has proven to be difficult to design satisfactory PAPG-based devices for BP estimation. Some previously-deployed PAPG-based devices for BP estimation include two spatially separated ultrasonic receivers for measuring pulse wave velocity (PWV) and a separate PAPG sensor element for measuring the cross-sectional area of an artery. However, providing both ultrasonic receivers in a single device with, for example, a smart watch form factor resulted in a limited ultrasonic receiver separation and error-prone PWV estimations. Providing a single device that had ultrasonic receivers spaced far enough apart for reasonably accurate PWV measurements required a large form factor, which was not acceptable for some consumers. Moreover, having a PAPG sensor element and 2 ultrasonic receivers in a single device leads to a somewhat cumbersome arrangement: for example, it is challenging to align all three sensor elements to a single artery in order to obtain accurate measurements of arterial diameter, arterial distension and PWV. Another challenge is the signal-to-noise ratio (SNR) for signals of interest, such as signals corresponding to ultrasound caused by the photoacoustic response of arterial walls, is relatively low. For example, the signals corresponding to arterial walls are generally significantly lower in amplitude than signals corresponding to the photoacoustic response of skin.

Some disclosed devices are configured to estimate both PWV and the cross-sectional area of an artery based on measurements taken at a single arterial location. Some such devices include a light source system, an ultrasonic receiver system and a control system. The light source system may be configured for providing light to a target object on an outer surface of the platen. The ultrasonic receiver system may be configured to receive ultrasonic waves generated by the target object responsive to the light from the light source system. According to some implementations, the ultrasonic receiver system may include a single array of ultrasonic receiver elements. In some implementations, the control system may be configured to control the light source system to provide light to the target object and to receive ultrasonic receiver signals from each of the ultrasonic receiver elements in the single array. The ultrasonic receiver signals may correspond to the ultrasonic waves generated by the target object responsive to the light. The control system may be configured to detect an artery within the target object and to estimate a BP within the artery based, at least in part, on ultrasonic receiver signals from the single array of M ultrasonic receiver elements.

Particular implementations of the subject matter described in this disclosure can be implemented to realize one or more of the following potential advantages. Some disclosed PAPG-based devices for BP estimation are capable of estimating—e.g., monitoring—BP according to single-sensor measurement. In some implementations, a single photoacoustic (PA) sensor may be configured to estimate both PWV and the area of an artery, thus enabling the monitoring of BP with measurements at a single arterial location. Accordingly, such devices include relatively less hardware than previously-deployed PAPG-based devices for BP estimation and have relatively less complexity and lower costs. Some disclosed PAPG-based devices for BP estimation do not rely on the methods for PWV estimation that were used by previously-disclosed devices and may provide more accurate measurements and BP estimates. The disclosed PAPG-based devices for BP estimation are suitable for compact form-factors, which is especially desirable for wearable devices such as smart watches or other wearable health-monitoring or fitness-monitoring devices.

According to some implementations, the control system may be configured to apply a receiver-side beamforming process to ultrasonic receiver signals received from each receiver element of the single array of ultrasonic receiver elements. In some implementations, the control system may be configured to estimate a change in cross-sectional area of the artery based, at least in part, on the beamformed ultrasonic receiver image. Such configurations include PAPG-capable devices that can provide a higher SNR—as compared to previously-deployed devices having a single receiver element—for signals corresponding to the photoacoustic response of one or more arterial walls. According to some implementations, the control system may be configured to estimate BP based, at least in part, on the change in the cross-sectional area of the artery. Signals obtained from multiple receiver elements of an ultrasonic receiver array can provide relatively more accurate information about the area of an artery that would not be available if, for example, signals were obtained from only a single ultrasonic receiver element.

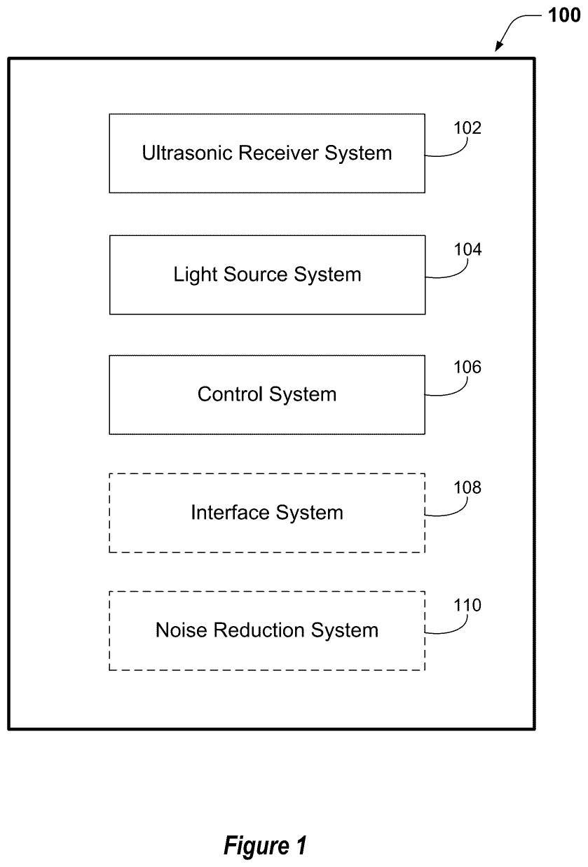

FIG. 1 is a block diagram that shows example components of an apparatus according to some disclosed implementations. In this example, the apparatus 100 includes an ultrasonic receiver system 102, a control system 106 and a light source system 104. According to some examples, the ultrasonic receiver system 102 and the light source system 104 may be co-located, to allow measurements to be obtained from a single arterial location. Some implementations of the apparatus 100 may include an interface system 108, a noise reduction system 110, or both. As with other disclosed implementations, in some alternative implementations the apparatus 100 may include more components, fewer components or different components.

In this example, the ultrasonic receiver system 102 is configured to receive ultrasonic waves generated by a target object responsive to the light from the light source system 104. According to some examples, the ultrasonic receiver system 102 includes an array of ultrasonic receiver elements. In some examples, the ultrasonic receiver system 102 includes a single array of M ultrasonic receiver elements. According to some examples, the single array of M ultrasonic receiver elements may be linearly arranged, or in other words arranged along a line. In some examples, the ultrasonic receiver system 102 may be, or may include, an array of electrodes arranged on a piezoelectric receiver layer, such as a layer of PVDF polymer, a layer of PVDF-TrFE copolymer, or a layer of piezoelectric composite material. In some implementations, other piezoelectric materials may be used in the piezoelectric layer, such as aluminum nitride (AlN) or lead zirconate titanate (PZT). The ultrasonic receiver system 102 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the ultrasonic receiver system 102 may be, or may include, an ultrasonic receiver array. In some examples, the apparatus 100 may include one or more separate ultrasonic transmitter elements. In some such examples, the ultrasonic transmitter(s) may include an ultrasonic plane-wave generator.

The light source system 104 may be configured to provide light to a target object on an outer surface of the apparatus 100. According to some implementations, the light source system 104 may include one or more light-emitting diodes (LEDs). In some implementations, the light source system 104 may include one or more laser diodes. According to some implementations, the light source system 104 may include one or more vertical-cavity surface-emitting lasers (VCSELs). In some implementations, the light source system 104 may include one or more edge-emitting lasers. In some implementations, the light source system may include one or more neodymium-doped yttrium aluminum garnet (Nd:YAG) lasers. The light source system 104 may, in some examples, include an array of N light sources, where N is an integer of 2 or more. The array of N light sources may be, or may include, an array of LEDs, an array of laser diodes, an array of VCSELs, an array of edge-emitting lasers, or combinations thereof.

According to some examples, the light source system 104 may include one or more light-directing elements configured to direct light from the light source system towards the target object along a first axis. In some examples, the one or more light-directing elements may include one or more light guides, which also may be referred to herein as light pipes. According to some examples, the one or more light-directing elements may include at least one diffraction grating. Alternatively, or additionally, the one or more light-directing elements may include at least one lens.

The light source system 104 may, in some examples, be configured to transmit light in one or more wavelength ranges. In some examples, the light source system 104 may configured for transmitting light in a wavelength range of 500 to 600 nanometers. According to some examples, the light source system 104 may configured for transmitting light in a wavelength range of 800 to 950 nanometers.

The light source system 104 may include various types of drive circuitry, depending on the particular implementation. In some disclosed implementations, the light source system 104 may include at least one multi-junction laser diode, which may produce less noise than single-junction laser diodes. In some examples, the light source system 104 may include a drive circuit (also referred to herein as drive circuitry) configured to cause the light source system to emit pulses of light at pulse widths in a range from 3 nanoseconds to 1000 nanoseconds. According to some examples, the light source system 104 may include a drive circuit configured to cause the light source system to emit pulses of light at pulse repetition frequencies in a range from 1 kilohertz to 100 kilohertz.

In some implementations, the light source system 104 may be configured for emitting various wavelengths of light, which may be selectable to trigger acoustic wave emissions primarily from a particular type of material. For example, because the hemoglobin in blood absorbs near-infrared light very strongly, in some implementations the light source system 104 may be configured for emitting one or more wavelengths of light in the near-infrared range, in order to trigger acoustic wave emissions from hemoglobin. However, in some examples the control system 106 may control the wavelength(s) of light emitted by the light source system 104 to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the ultrasonic receiver system 102. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying RGDs) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system 104 may be configured for emitting a light pulse with a pulse width less than about 100 nanoseconds. In some implementations, the light pulse may have a pulse width between about 10 nanoseconds and about 500 nanoseconds or more. According to some examples, the light source system may be configured for emitting a plurality of light pulses at a pulse repetition frequency between 10 Hz and 100 kHz. Alternatively, or additionally, in some implementations the light source system 104 may be configured for emitting a plurality of light pulses at a pulse repetition frequency between about 1 MHz and about 100 MHz. Alternatively, or additionally, in some implementations the light source system 104 may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and the substrate. For example, a set of four or more light pulses may be emitted from the light source system 104 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system 104. In some implementations, the light source system may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

The control system 106 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 106 also may include (and/or be configured for communication with) one or more memory devices, such as one or more random access memory (RAM) devices, read-only memory (ROM) devices, etc. Accordingly, the apparatus 100 may have a memory system that includes one or more memory devices, though the memory system is not shown in FIG. 1. The control system 106 may be configured for receiving and processing data from the ultrasonic receiver system 102, e.g., as described below. If the apparatus 100 includes an ultrasonic transmitter, the control system 106 may be configured for controlling the ultrasonic transmitter. In some implementations, functionality of the control system 106 may be partitioned between one or more controllers or processors, such as a dedicated sensor controller and an applications processor of a mobile device.

In some examples, the control system 106 may be configured to control the light source system 104 to emit light towards a target object on an outer surface of the apparatus 100. In some such examples, the control system 106 may be configured to receive ultrasonic receiver signals from each of a plurality of ultrasonic receiver elements in a single array of M ultrasonic receiver elements of the ultrasonic receiver system 102, where M is an integer of two or more. The ultrasonic receiver signals may correspond to the ultrasonic waves generated by the target object responsive to the light from the light source system 104.

According to some examples, the control system 106 may be configured to detect an artery within the target object based on the ultrasonic receiver signals. In some examples, the control system 106 may be configured to estimate a blood pressure within the artery based on ultrasonic receiver signals from the single array of M ultrasonic receiver elements.

In some examples, the control system 106 may be configured to apply a receiver-side beamforming process to the ultrasonic receiver signals, to produce a beamformed ultrasonic receiver image. According to some examples, the control system 106 may be configured to estimate a change in cross-sectional area of the artery based, at least in part, on the beamformed ultrasonic receiver image. In some such examples, the control system 106 may be configured to estimate blood pressure based, at least in part, on the change in the cross-sectional area of the artery.

Some implementations of the apparatus 100 may include the interface system 108. In some examples, the interface system 108 may include a wireless interface system. In some implementations, the interface system 108 may include a user interface system, one or more network interfaces, one or more interfaces between the control system 106 and a memory system and/or one or more interfaces between the control system 106 and one or more external device interfaces (e.g., ports or applications processors), or combinations thereof. According to some examples in which the interface system 108 is present and includes a user interface system, the user interface system may include a microphone system, a loudspeaker system, a haptic feedback system, a voice command system, one or more displays, or combinations thereof. According to some examples, the interface system 108 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

According to some examples, the apparatus 100 may include a noise reduction system 110. For example, the noise reduction system 110 may include one or more mirrors that are configured to reflect light from the light source system 104 away from the ultrasonic receiver system 102. In some implementations, the noise reduction system 110 may include one or more sound-absorbing layers, acoustic isolation material, light-absorbing material, light-reflecting material, or combinations thereof. In some examples, the noise reduction system 110 may include acoustic isolation material, which may reside between the light source system 104 and at least a portion of the ultrasonic receiver system 102, on at least a portion of the ultrasonic receiver system 102, or combinations thereof. In some examples, the noise reduction system 110 may include one or more electromagnetically shielded transmission wires. In some such examples, the one or more electromagnetically shielded transmission wires may be configured to reduce electromagnetic interference from circuitry of the light source system 104, receiver system circuitry, or combinations thereof, that is received by the ultrasonic receiver system 102. In some examples, the one or more electromagnetically shielded transmission wires, sound-absorbing layers, acoustic isolation material, light-absorbing material, light-reflecting material, or combinations thereof may be components of the ultrasonic receiver system 102, the light source system 104, or both. Despite the fact that the ultrasonic receiver system 102, the light source system 104 and the noise reduction system 110 are shown in FIG. 1 as being separate elements, such components may nonetheless be regarded as elements of the noise reduction system 110.

The apparatus 100 may be used in a variety of different contexts, some examples of which are disclosed herein. For example, in some implementations a mobile device, such as a hand-held device, may include the apparatus 100. In some implementations, a wearable device may include the apparatus 100. The wearable device may, for example, be a bracelet, an armband, a wristband, a watch, a ring, a headband or a patch. Accordingly, in some examples the apparatus 100 may be configured to be worn by, or attached to, a person.

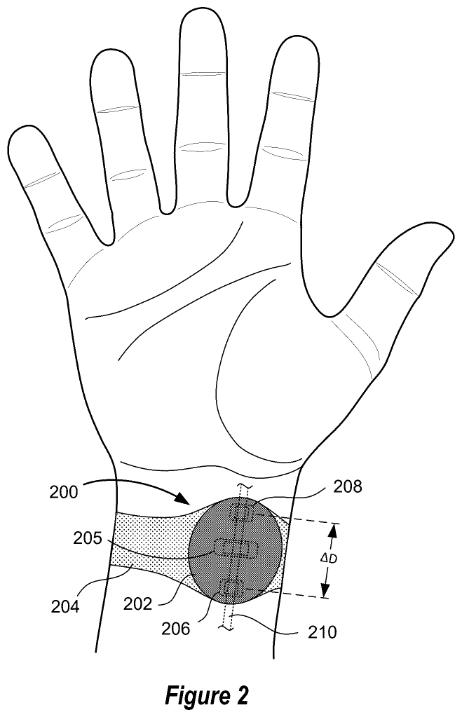

FIG. 2 shows an example of a previously-deployed PAPG-based device for BP estimation. In this example, the monitoring device 200 is designed to be worn around a human wrist. In the illustrated example, the monitoring device 200 includes a housing 202 that is integrally formed with, coupled to or otherwise integrated with a wristband 204. The sensors 206 and 208 may, in some instances, each include portions of an ultrasonic receiver system. The sensor 205 may, in some instances, include a portion of an ultrasonic receiver system and an instance of a light source system. In this example, the monitoring device 200 is coupled around the wrist such that the sensors 205, 206 and 208 within the housing 202 are each positioned along a segment of the radial artery 210 (note that the sensors are generally hidden from view from the external or outer surface of the housing facing the subject—in this example, a portion of a person's wrist—while the monitoring device is coupled with the subject, but exposed on an inner surface of the housing to enable the sensors to obtain measurements from the underlying artery through the subject's skin).

According to this example, the sensors 206 and 208 are configured to measure PWV of blood within the radial artery 210, whereas the sensor 205 is configured to measure changes in the cross-sectional area of the radial artery 210. Based at least in part on these measurements, the monitoring device 200 is designed to estimate blood pressure.

As shown in FIG. 2, the sensors 206 and 208 are separated by a fixed distance ΔD. As noted elsewhere herein, with a relatively small ΔD between two ultrasonic receiver portions that is feasible for a single device with a smart watch form factor has resulted error-prone PWV estimations. Providing a single device that had ultrasonic receivers spaced far enough apart for reasonably accurate PWV measurements required a large form factor, which was not acceptable for some consumers. Moreover, BP estimation should be based on accurate measurements from the same artery. Having a PAPG sensor element—such as the sensor 205—and 2 ultrasonic receivers with a sufficiently large ΔD for accurate PWV determination in a single wearable device causes potential challenges with aligning all three sensor elements along a single artery.

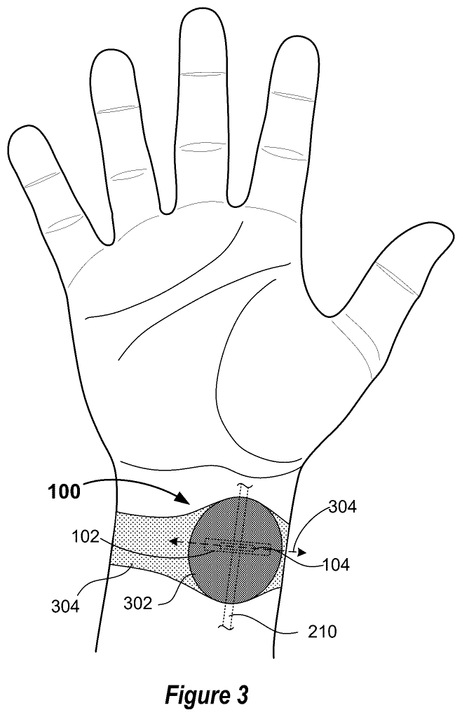

FIG. 3 shows an example of a PAPG-based device for BP estimation according to the present disclosure. The apparatus 100 of FIG. 3 is an instance of the apparatus 100 of FIG. 1. Although not shown in FIG. 3, the apparatus 100 includes an instance of the control system 106. In some instances, the apparatus 100 may include an interface system 108, a noise reduction system 110, or combinations thereof. As with other disclosed examples, the type, number, size and arrangement of elements shown in FIG. 3 and described herein are merely examples. Other implementations may include different types of elements, numbers of elements, arrangements of elements, or combinations thereof.

In this example, the apparatus 100 is designed to be worn around a human wrist and includes a housing 302 that is integrally formed with, coupled to or otherwise integrated with a wristband 304. According to this example, the outward appearance of the apparatus 100 is like that of the monitoring device 200 of FIG. 2. However, instead of having separate sensors 205, 206 and 208, as shown in the example of FIG. 2, the apparatus 100 includes co-located instances of the ultrasonic receiver system 102 and the light source system 104. In this example, the co-located instances of the ultrasonic receiver system 102 and the light source system 104 are integrated into a single photoacoustic (PA) sensor that is configured to estimate both PWV and the area of the radial artery 210—including changes in the area of the radial artery 210—thus enabling the monitoring of BP with measurements at a single arterial location. Taking measurements at a single arterial location can improve the accuracy of such measurements and therefore can improve the accuracy of corresponding BP estimations.

According to some examples, the ultrasonic receiver system 102 may include a single array of M ultrasonic receiver elements, M being an integer of 2 or more. In some instances, M may be 2, 3, 4, 5, 6, 7, 8, 9, 10, 11, 12, etc. In some examples, the single array of M ultrasonic receiver elements may extend along an axis 304 that is configured to extend across the radial artery 210. One could consider the “single arterial location” at which measurements are taken to be the intersection of the axis 304 and the radial artery 210. According to some examples, the light source system 104 may include N light sources, wherein N is an integer of 1 or more. In some instances, N may be 1, 2, 3, 4, 5, 6, 7, 8, etc. In some examples, when N is 2 or more, the N light sources also may extend along the axis 304.

The novel apparatus 100 shown in FIG. 3 is made possible, at least in part, by different measurement and BP calculation techniques that are disclosed herein. Unlike previously-deployed PAPG-based devices for BP estimation, the apparatus 100 does not require two separate ultrasonic receiver portions for PWV estimations. Accordingly, the apparatus 100 includes relatively less hardware than previously-deployed PAPG-based devices for BP estimation, has relatively less complexity and may potentially be manufactured at a lower cost. As described in more detail below, the apparatus 100 does not rely on the methods for PWV estimation that were used by previously-disclosed devices and may provide more accurate measurements and BP estimates. The apparatus 100 is suitable for compact form-factors, which is highly desirable for wearable devices such as smart watches or other wearable health-monitoring or fitness-monitoring devices. In some other implementations, the apparatus 100 may be designed or adapted for positioning around a forearm, an upper arm, an ankle, a lower leg, an upper leg, an ear or a finger using one or more straps, bands, etc. In some alternative implementations, the apparatus 100 may be implemented in a wearable patch, which in some examples may be adhesively attached to a portion of the human body.

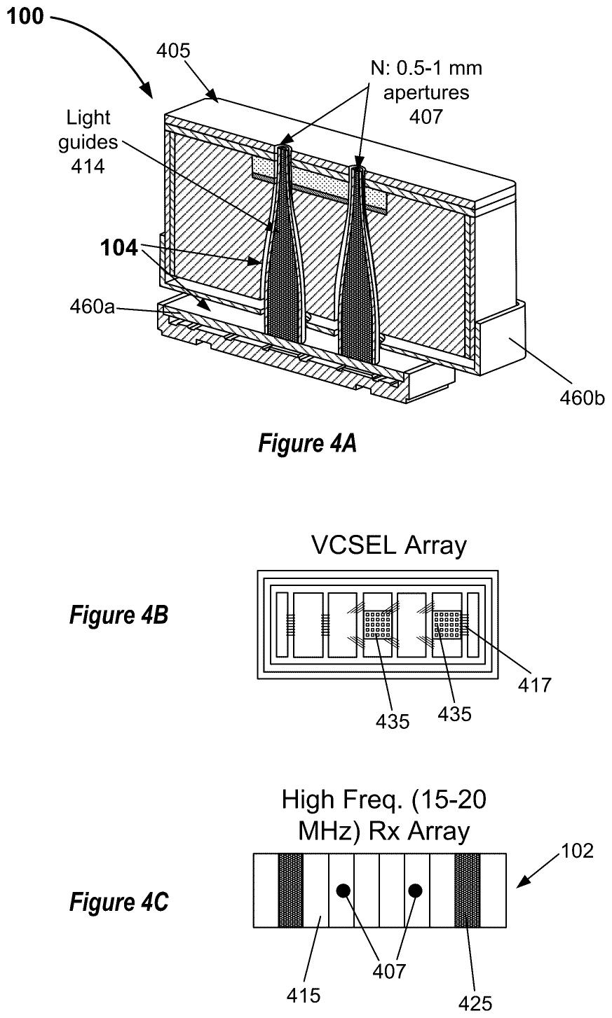

FIG. 4A shows an example of an ultrasonic receiver system and a light source system integrated into a single photoacoustic (PA) sensor. FIG. 4B shows an example of the light source system of FIG. 4A. FIG. 4C shows an example of the ultrasonic receiver system of FIG. 4A. The apparatus 100 of FIG. 4A is an instance of the apparatus 100 of FIG. 1 and is an example of the co-located ultrasonic receiver system 102 and light source system 104 shown in FIG. 3. Although not shown in FIG. 4A, the apparatus 100 includes an instance of the control system 106. In some instances, the apparatus 100 may include an interface system 108, a noise reduction system 110, or combinations thereof. As with other disclosed examples, the types, numbers, sizes and arrangements of elements shown in FIGS. 4A-4C and described herein are merely examples. Other implementations may include different types of elements, numbers of elements, arrangements of elements, or combinations thereof.

According to this example, the ultrasonic receiver system 102 of FIG. 4C includes a single array of M active ultrasonic receiver elements 415 and two inactive ultrasonic receiver elements 425. In this example, M=6. In some alternative examples, M may be 2, 3, 4, 5, 7, 8, 9, 10, 11, 12, etc. In some examples, the apparatus 100 may be configured such that the single array of M active ultrasonic receiver elements may extend along an axis that is configured to extend across an artery, such as the radial artery 210 that is shown in FIG. 3. According to this example, two of the active ultrasonic receiver elements 415 each include one of the apertures 407, which are configured to allow the light guides 440 to pass through the ultrasonic receiver system 102.

In this example, the light source system 104 of FIGS. 4A and 4B includes N light guides 440, light source system circuitry 417 and N light-emitting components 435, which are VCSELs in this example. The N light guides 440 and the N light-emitting components 435, taken together, are examples of N light sources. In this example, N=2. According to some alternative examples, N may be 1, 3, 4, 5, 6, 7, 8, etc. In some examples, the apparatus 100 may be configured such that an array of N active ultrasonic According to this example, the light guides 440 are configured to convey light from the light-emitting components 435 through the apparatus 100, including the ultrasonic receiver system 102 and the surface 405 via the apertures 407, to be emitted from the apparatus 100. The apparatus 100 may, in some implementations, be configured such that the surface 405 will reside proximate a portion of a human body—such as a wrist, a finger, a leg, etc.—when the apparatus 100 is worn. The light source system circuitry 417 may be configured to electrically connect the light-emitting components 435 with at least a portion of the control system 106.

As noted elsewhere herein, some implementations of the apparatus 100 may include a noise reduction system 110. One type of noise that may be present in the apparatus 100 involves electromagnetic interference (EMI) from the light source system circuitry 417 that may be received by the receiver system 102. In some examples, the apparatus 100 may include one or more EMI-reducing elements that reside between the light source system circuitry 417 and the receiver system 102. According to some such examples, the material 460a of FIG. 4A, the material 460b of FIG. 4A, or both, may be, or may include, one or more types of EMI shielding material. In some examples, EMI-reducing material may surround the light source system circuitry 517. Instances of such EMI-reducing material may be regarded as components of the noise reduction system 110 of FIG. 1.

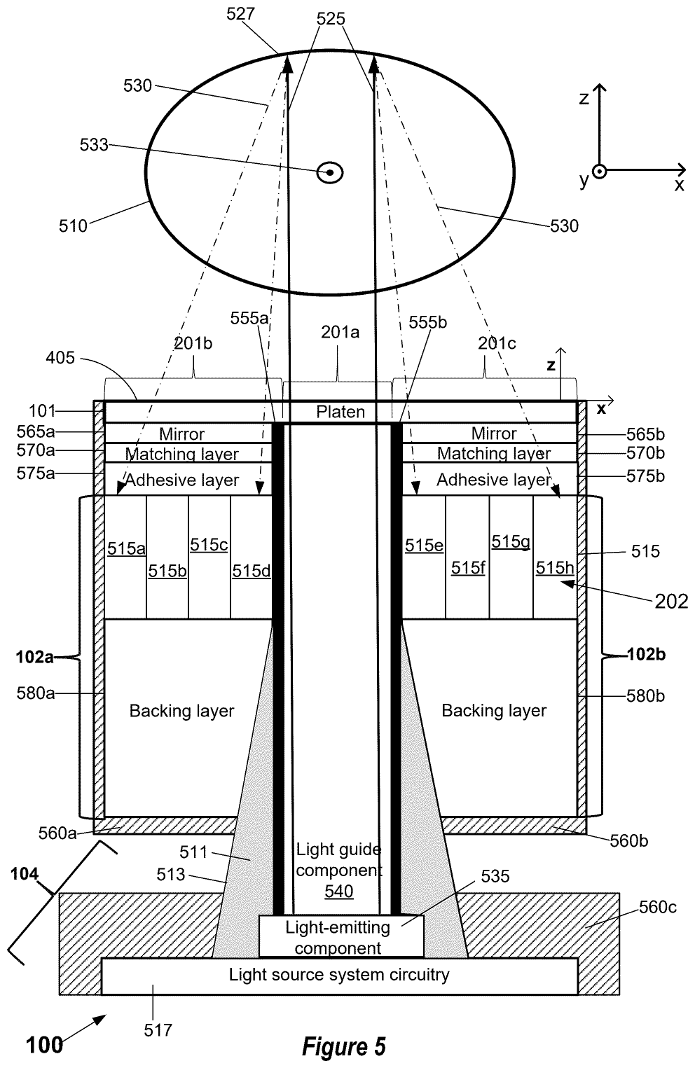

FIG. 5 shows another example of an ultrasonic receiver system and a light source system integrated into a single photoacoustic (PA) sensor. As with other figures provided herein, the numbers, types and arrangements of elements shown in FIG. 5 are merely presented by way of example. In this example, the apparatus 100 is an instance of the apparatus 100 shown in FIG. 1.

In this example, the receiver stack portion 102a includes ultrasonic receiver elements 515a, 515b, 515c and 515d, as well as the backing layer 580a. Here, the receiver stack portion 102b includes ultrasonic receiver elements 515e, 515f, 515g and 515h, as well as the backing layer 580b. According to this implementation, the apparatus 100 includes a platen 101 having a platen portion 201a proximate the light guide component 540, a platen portion 201b proximate the ultrasonic receiver elements 515a-515d and a platen portion 201c proximate the ultrasonic receiver elements 515e-515h. In this example, the ultrasonic receiver elements 515a-515d and the ultrasonic receiver elements 515e-515h are elements of a single array of M ultrasonic receiver elements 515a-515h, where M=8. According to this example, the array of ultrasonic receiver elements 515a-515h extends along the x axis. FIG. 5 is not drawn to scale. One of ordinary skill in the art will understand that although the light guide component 540 is shown in FIG. 5 as having a width equal to the widths of about three of the ultrasonic receiver elements, the light guide component 540—or at least the portion of the light guide component 540 that passes through the ultrasonic receiver elements 515a-515h—may actually be narrower than a single ultrasonic receiver element, e.g., as shown in FIG. 4. In some alternative implementations, the ultrasonic receiver elements 515a and 515h may be part of the same ultrasonic receiver element ring. In some such implementations, the ultrasonic receiver elements 515b and 515g may be part of a second ultrasonic receiver element ring, the ultrasonic receiver elements 515c and 515f may be part of a third ultrasonic receiver element ring and the ultrasonic receiver elements 515d and 515e may be part of a fourth ultrasonic receiver element ring. In still other alternative implementations, the ultrasonic receiver elements 515a-515h may be part of an two-dimensional array of ultrasonic receiver elements that extends along the x axis and the y axis.

According to the example shown in FIG. 5, the apparatus 100 includes a light-coupling component 511, which is configured to couple light from the light-emitting component 535 into the light guide component 540. According to this example, the number of light sources N=1. In this example, the light-coupling component 511 has an outer surface 513 that is frustum-shaped. According to this example, the light-coupling component 511 resides partially between the receiver stack portions 102a and 102b and the light guide component 540, in order to reduce the overall thickness of the apparatus 100 along the z axis. Although some other disclosed implementations are not shown to have a light-coupling component, alternative examples of such disclosed implementations may, in fact, include one or more light-coupling components.

In this example, the light source system is transmitting light 525 into an artery portion 510 that has an axis 533 that is substantially parallel to (e.g., within 5 degrees of being parallel, within 10 degrees of being parallel, within 15 degrees of being parallel, etc.) the y axis and therefore is substantially perpendicular to the axis along which the linear array of ultrasonic receiver elements 515a-515h is arranged. According to this example, some of the light 525 has reached the arterial wall 527 and has caused the arterial wall 527 to emit the photoacoustic waves 530, which are received by the linear array of ultrasonic receiver elements 515a-515h.

The backing layers 580a and 580b may be configured to suppress at least some acoustic artifacts and may provide a relatively higher signal-to-noise ratio (SNR) than receiver systems 102 that lack a backing layer. In some examples, the backing layers 580a and 580b may include metal, epoxy, or a combination thereof.

As described elsewhere herein, some implementations of the apparatus 100 include one or more elements configured for noise reduction. These noise reduction elements may be considered to be part of the noise reduction system 110 that is described with reference to FIG. 1. However, such noise reduction elements may reside in various parts of the apparatus 100.

One type of noise that may be present in the apparatus 100 involves the leakage of light from the light source system 104, such as from the light guide component 540 of FIG. 5, to the receiver system 102. According to the example shown in FIG. 5, the apparatus 100 includes the light-mitigating element 555a, which resides between the light guide component 540 and the receiver stack portion 102a, and the light-mitigating element 555b, which resides between the light guide component 540 and the receiver stack portion 102b. In this example, the light-mitigating elements 555a and 555b also reside between the light guide component 540 and the mirror layers 565a and 565b, the matching layers 570a and 570b, and the adhesive layers 575a and 575b. In some examples, the light-mitigating elements 555a and 555b may be discrete elements, whereas in other examples the light-mitigating elements 555a and 555b may be portions of a continuous element, such as a cylinder that surrounds the light guide component 540. In some examples, the light-mitigating elements 555a and 555b may include material having a relatively low index of refraction, such as a low refractive index foam. In this example, the light-mitigating elements 555a and 555b are configured to increase optical coupling and reduce optical losses.

Another type of noise that may be present in the apparatus 100 involves EMI from the light source system circuitry 517 that may be received by the receiver system 102. In the example shown in FIG. 5, the apparatus 100 includes the EMI-reducing element 560a proximate the receiver stack portion 102a and the EMI-reducing element 560b proximate the receiver stack portion 102b. In some examples, the EMI-reducing elements 560a and 560b may include one or more types of EMI shielding material. According to some examples, the EMI-reducing elements 560a and 560b may be portions of a continuous element, such as a cylinder. In this example, the EMI-reducing element 560c resides between the light source system circuitry 517 and the receiver stack portions 102a and 102b. In some examples, the EMI-reducing element 560c may surround the light source system circuitry 517.

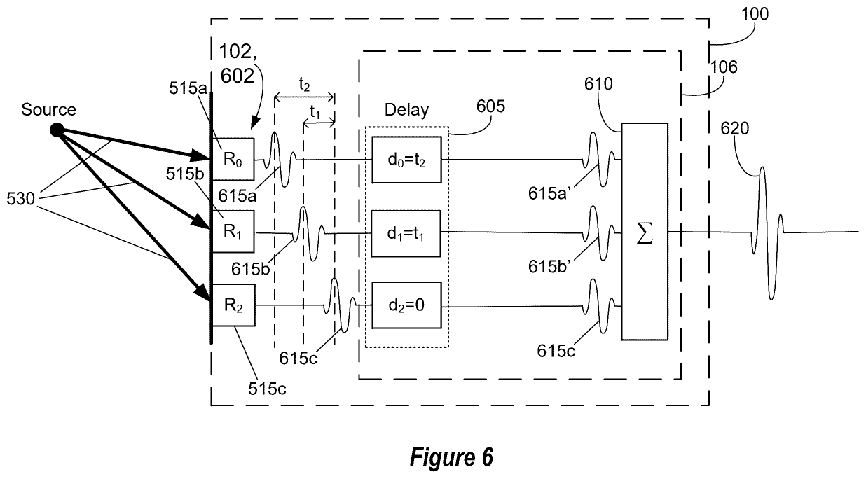

FIG. 6 shows an example of an apparatus that is configured to perform a receiver-side beamforming process. In this example, the receiver-side beamforming process is a delay-and-sum beamforming process. As with other disclosed examples, the types, numbers, sizes and arrangements of elements shown in FIG. 6 and described herein, as well as the associated described methods, are merely examples.

In this example, a source is shown emitting ultrasonic waves 530, which are detected by active ultrasonic receiver elements 515a, 515b and 515c of an array of ultrasonic receiver elements 602. The array of ultrasonic receiver elements 602 is part of an ultrasonic receiver system 102. The ultrasonic waves 530 may, in some examples, correspond to the photoacoustic response of a target object to light emitted by a light source system 104 of the apparatus 101. In this example, the active ultrasonic receiver elements 515a, 515b and 515c provide ultrasonic receiver signals 615a, 615b and 615c, respectively, to the control system 106.

According to this example, the control system 106 includes a delay module 605 and a summation module 610. In this example, the delay module 605 is configured to determine whether a delay should be applied to each of the ultrasonic receiver signals 615a, 615b and 615c, and if so, what delay to apply. According to this example, the delay module 605 determines that a delay d₀ of t₂ should be applied to the ultrasonic receiver signal 615a, that a delay d₁ of t₁ should be applied to the ultrasonic receiver signal 615b and that no delay should be applied to the ultrasonic receiver signal 615c. Accordingly, the delay module 605 applies a delay of t₂ to the ultrasonic receiver signal 615a, producing the ultrasonic receiver signal 615a′, and applies a delay of t₁ to the ultrasonic receiver signal 615b, producing the ultrasonic receiver signal 615b′.

In some examples, the delay module 605 may determine what delay, if any, to apply to an ultrasonic receiver signal by performing a correlation operation on input ultrasonic receiver signals. For example, the delay module 605 may perform a correlation operation on the ultrasonic receiver signals 615a and 615c, and may determine that by applying a time shift of t₂ to the ultrasonic receiver signal 615a, the ultrasonic receiver signal 615a would be strongly correlated with the ultrasonic receiver signal 615c. Similarly, the delay module 605 may perform a correlation operation on the ultrasonic receiver signals 615b and 615c, and may determine that by applying a time shift of t₁ to the ultrasonic receiver signal 615b, the ultrasonic receiver signal 615b would be strongly correlated with the ultrasonic receiver signal 615c.

According to this example, the summation module 610 is configured to sum the ultrasonic receiver signals 615a′, 615b′ and 615c, producing the summed signal 620. One may observe that the amplitude of the summed signal 620 is greater than the amplitude of any one of the ultrasonic receiver signals 615a, 615b or 615c. In some instances, the signal-to-noise ratio (SNR) of the summed signal 620 may be greater than the SNR of any of the ultrasonic receiver signals 615a, 615b or 615c.

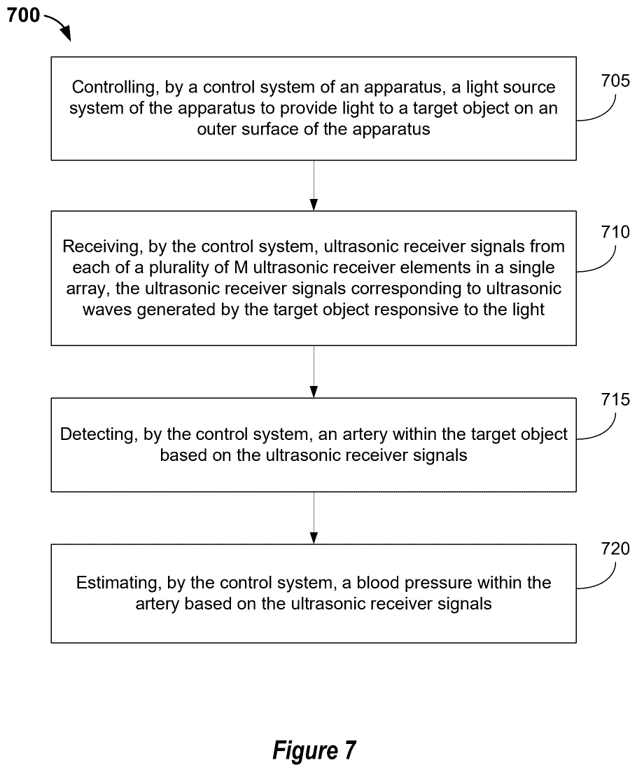

FIG. 7 is a flow diagram that shows examples of some disclosed operations. The blocks of FIG. 7 may, for example, be performed by the apparatus 100 of any one of FIG. 1, 3, 4 or 5, or by a similar apparatus. As with other methods disclosed herein, the method outlined in FIG. 7 may include more or fewer blocks than indicated. Moreover, the blocks of methods disclosed herein are not necessarily performed in the order indicated. In some instances, one or more of the blocks shown in FIG. 7 may be performed concurrently.

In this example, block 705 involves controlling, by a control system of an apparatus, a light source system of the apparatus to provide light to a target object on an outer surface of the apparat. The target object may be a portion of a finger, a wrist, an arm, a leg, etc., depending on the particular example. The control system may be an instance of the control system 106 and the light source system may be an instance of the light source system 104. In some examples, the light source system may include N light sources. In some instances, N may be 1, 2, 3, 4, 5, 6, 7, 8, etc. In some examples, the N light sources may include two or more vertical-cavity surface-emitting lasers (VCSELs).

According to this example, block 710 involves receiving, by the control system, ultrasonic receiver signals from each of M ultrasonic receiver elements in a single array. In some examples, M may be an integer of 2 or more. In some instances, M may be 2, 3, 4, 5, 6, 7, 8, 9, 10, 11, 12, etc. In this example, the ultrasonic receiver signals correspond to ultrasonic waves generated by the target object responsive to the light from the light source system. The ultrasonic receiver elements may be elements of the ultrasonic receiver system 102, such as the ultrasonic receiver elements 415 of FIG. 4 or the ultrasonic receiver elements 515a-515h of FIG. 5. In some examples, the ultrasonic receiver signals may correspond to ultrasonic waves generated by an arterial wall—such as the arterial wall 527 that is shown in FIG. 5—to blood within an artery, or both.

According to this example, block 715 involves detecting, by the control system, an artery within the target object based on the ultrasonic receiver signals. In some examples, the artery may be a radial artery, such as the radial artery 210 of FIG. 3. According to some such examples, the apparatus may be configured to be worn on a human wrist, as in the example of the apparatus 100 of FIG. 3. In other examples, the artery may be another type of artery, such as an artery inside an upper arm, a leg, a neck, an ear, etc. In some examples, detecting the artery may involve applying image recognition software, for example software that is trained to detect ultrasonic receiver signals corresponding to arterial walls, ultrasonic receiver signals corresponding to blood within an artery, etc. In some examples, detecting the artery may involve applying a range gate delay (RGD) corresponding to a likely arterial depth within the target object, a range gate window (RGW) corresponding to a likely range of arterial depths within the target object, etc.

In this example, block 720 involves estimating, by the control system, a blood pressure within the artery based on ultrasonic receiver signals from the single array of M ultrasonic receiver elements. According to some examples, method 700 may involve estimating a change in cross-sectional area of the artery based at least in part the ultrasonic receiver signals from the single array of M ultrasonic receiver elements. In some such examples, estimating the blood pressure may be based, at least in part, on the change in the cross-sectional area of the artery. In some examples, method 700 may involve applying, by the control system, a receiver-side beamforming process to the ultrasonic receiver signals from the single array of M ultrasonic receiver elements, to produce a beamformed ultrasonic receiver image. In some such examples, estimating a cross-sectional area of the artery, a change in the cross-sectional area of the artery, or both, may be based at least in part on the beamformed ultrasonic receiver image.

According to some examples, method 700 may involve estimating, by the control system, the change in the blood flow rate within the artery based on measurements from an ultrasonic receiver system and a light source system integrated into a single photoacoustic (PA) sensor. In some such examples, method 700 may involve estimating, by the control system, the change in the blood flow rate within the artery based on measurements from a co-located ultrasonic receiver system 102 and light source system 104, such as that shown in one of FIGS. 3-5. In some examples, method 700 may involve estimating a change in a blood flow rate within the artery based on the ultrasonic receiver signals from the single array of M ultrasonic receiver elements. In some such examples, the single array of M ultrasonic receiver elements may be linearly arranged. In some such examples, estimating the blood pressure may be based, at least in part, on the change in the blood flow rate within the artery. Accordingly, in some examples, estimating the blood pressure may be based at least in part on the change in the blood flow rate within the artery and the change in cross-sectional area of the artery.

According to some examples, the ultrasonic receiver signals may include frequencies in a range from 10 MHz to 25 MHz. According to some examples, the control system may be configured to receive ultrasonic receiver signals from the ultrasonic receiver system at a frame rate in a range from 1 KHz to 3 KHz. Such frequencies and frame rates can provide sufficiently accurate measurements of the blood flow rate within the artery, and the change in the blood flow rate within the artery, for reliable BP estimation.

In some examples, method 700 may involve estimating, by the control system, the change in the blood flow rate within the artery based at least in part on speckle decorrelation-based velocimetry imaging. Some such examples may involve applying one or more methods disclosed in R. Zangabad et al., “Photoacoustic flow velocity imaging based on complex field decorrelation,” (Photoacoustics, Volume 22, June 2021, 100256), which is hereby incorporated by reference and for all purposes.

In some alternative examples, method 700 may involve estimating, by the control system, the change in the blood flow rate within the artery based at least in part on a structured-illumination photoacoustic doppler method. Some such alternative examples may involve applying one or more methods disclosed in R. Zhang et al. “Structured-illumination photoacoustic Doppler flowmetry of axial flow in homogeneous scattering media” (Appl. Phys. Lett. 103, 094101 ((2013)), which is hereby incorporated by reference and for all purposes.

According to some alternative examples, method 700 may involve estimating, by the control system, the change in the blood flow rate within the artery based at least in part on cross-correlation-based flow imaging in the time domain. Some such alternative examples may involve applying one or more methods disclosed in J. Brunker and P. Beard, “Pulsed photoacoustic Doppler flowmetry using time-domain cross-correlation: Accuracy, resolution and scalability,” (J. Acoust. Soc. Am. 132, 1780-1791 (2012), which is hereby incorporated by reference and for all purposes.

In some alternative examples, method 700 may involve estimating, by the control system, the change in the blood flow rate within the artery based at least in part on a continuous wave photoacoustic doppler method, or on cross-correlation-based flow imaging in a spatial domain.

In some implementations, the apparatus performing the method 700 may include a magnetic sensor system. According to some such examples, method 700 may involve estimating a change in a blood flow rate within the artery based on magnetic sensor signals from the magnetic sensor system. In some implementations, the apparatus performing the method 700 may include an optical sensor system. In some such examples, method 700 may involve estimating a change in a blood flow rate within the artery based on optical sensor signals from the optical sensor system.

As noted above, estimating the blood pressure may, in some examples, be based at least in part on the change in the blood flow rate within the artery and the change in cross-sectional area of the artery. In some such examples, estimating the blood pressure may be based in part on a derivative of the blood flow rate (Q) within the artery with respect to the cross-sectional area (A) of the artery. This derivative may be expressed as dQ/dA.

Following is a brief explanation of some underlying theories that have been developed by the present inventors in which BP estimation is based, in part, on dQ/dA.

According to the Bramwell-Hill equation, pulse wave velocity (PWV) is directly related to compliance, as follows:

$$PWV = \sqrt{\frac{A}{ρ} \frac{d P}{dA}} , \qquad ( 1 )$$

In Equation (1), A represents the cross-sectional area of an artery, P represents the blood pressure in the artery, and ρ represents the density of blood in the artery. We can rewrite Equation (1) to generate a pressure waveform—assuming PWV is relatively constant during a cardiac cycle—as follows:

$$P ( t ) - {P}_{0} = ρ {PWV}^{2} ln ( \frac{A ( t )}{{A}_{0}} ) , \qquad ( 2 )$$

In Equation (2), P₀ represents the blood pressure at arterial area Ao.

Some disclosed “flow/area” methods involve directly estimating a local PWV based on the change of blood flow rate with respect to the change in arterial area during a cardiac cycle. The characteristic impedance of a blood vessel, such as an artery, may be expressed as follows:

$${Z}_{c} = \frac{d P}{dQ} = \sqrt{\frac{ρ}{A} \frac{d P}{dA}} , \qquad ( 3 )$$

In Equation (3), Q represents the blood flow rate within a blood vessel.

By combining Equations (1) and (3), PWV may be expressed as follows:

$${PWV}_{QA} = \sqrt{\frac{A}{ρ} \frac{d P}{dA}} = \frac{1}{{Z}_{c}} \frac{d P}{dA} = \frac{dQ}{d P} \frac{d P}{dA} = \frac{dQ}{d A} . \qquad ( 4 )$$

We have disclosed various methods for calculating the how the area of a blood vessel varies during the cardiac cycle. Such methods can be used to obtain dA. For example, as noted elsewhere herein, in some examples method 700 may involve applying, by the control system, a receiver-side beamforming process to the ultrasonic receiver signals from the single array of M ultrasonic receiver elements, to produce a beamformed ultrasonic receiver image. In some such examples, estimating a cross-sectional area of the artery, a change in the cross-sectional area of the artery, or both, may be based at least in part on the beamformed ultrasonic receiver image. Accordingly, in some such examples, dA may be based, at least in part, on beamformed ultrasonic receiver images of an arterial cross-sectional area.

Similarly, we have disclosed various methods for calculating how the rate of blood flow in a blood vessel varies during the cardiac cycle based on measurements at a single arterial location using an ultrasonic receiver system and a light source system integrated into a single photoacoustic (PA) sensor. These methods include, but are not limited to, including speckle decorrelation-based velocimetry imaging, a continuous wave photoacoustic doppler method, a structured-illumination photoacoustic doppler method, cross-correlation-based flow imaging in a time domain or cross-correlation-based flow imaging in a spatial domain. Such methods can be used to obtain dQ.

After dA and dQ are obtained, PWV may be estimated using Equation 4 according to some examples. According to some such examples, after PWV has been estimated, BP may be estimated using Equation (2) along with measurements of A(t), which represents how the arterial area varies over time.

Implementation examples are described in the following numbered clauses:
1. An apparatus, including: a light source system configured to provide light to a target object on an outer surface of the apparatus, the light source system including N light sources; an ultrasonic receiver system configured to receive ultrasonic waves generated by the target object responsive to the light from the light source system, the ultrasonic receiver system including a single array of M ultrasonic receiver elements; and a control system configured to: control the light source system to provide light to the target object; receive ultrasonic receiver signals from each of a plurality of the M ultrasonic receiver elements in the single array, the ultrasonic receiver signals corresponding to the ultrasonic waves generated by the target object responsive to the light; detect an artery within the target object based on the ultrasonic receiver signals; and estimate a blood pressure within the artery based on the ultrasonic receiver signals.
2. The apparatus of clause 1, where the apparatus is configured to be worn on a human wrist and where the artery is a radial artery.
3. The apparatus of clause 1 or clause 2, where the control system is further configured to apply a receiver-side beamforming process to the ultrasonic receiver signals, to produce a beamformed ultrasonic receiver image.
4. The apparatus of clause 3, where: the control system is further configured to estimate a change in cross-sectional area of the artery based at least in part on the beamformed ultrasonic receiver image; and estimating the blood pressure is based in part on the change in the cross-sectional area of the artery.
5. The apparatus of clause 4, where: the control system is further configured to estimate a change in a blood flow rate within the artery based on the ultrasonic receiver signals; and estimating the blood pressure is based in part on the change in the blood flow rate within the artery and the change in cross-sectional area of the artery.
6. The apparatus of clause 5, where estimating the blood pressure is based in part on a derivative of the blood flow rate (Q) within the artery with respect to the cross-sectional area (A) of the artery (dQ/dA).
7. The apparatus of clause 6, where the control system is configured to estimate a pulse wave velocity based on dQ/dA.
8. The apparatus of clause 5 or clause 6, where the control system is configured to estimate the change in the blood flow rate within the artery based at least in part on speckle decorrelation-based velocimetry imaging, a continuous wave photoacoustic doppler method, a structured-illumination photoacoustic doppler method, cross-correlation-based flow imaging in a time domain or cross-correlation-based flow imaging in a spatial domain.
9. The apparatus of any one of clauses 4-7, further including a magnetic sensor system, where: the control system is further configured to estimate a change in a blood flow rate within the artery based on magnetic sensor signals from the magnetic sensor system; and estimating the blood pressure is based in part on the change in the blood flow rate within the artery and the change in cross-sectional area of the artery.
10. The apparatus of any one of clauses 4-7, further including an optical sensor system, where: the control system is further configured to estimate a change in a blood flow rate within the artery based on optical sensor signals from the optical sensor system; and estimating the blood pressure is based in part on the change in the blood flow rate within the artery and the change in cross-sectional area of the artery.
11. The apparatus of any one of clauses 1-10, where the control system is configured to receive ultrasonic receiver signals from the ultrasonic receiver system at a frame rate in a range from 1 KHz to 3 KHz.
12. The apparatus of any one of clauses 1-11, where the ultrasonic receiver signals include frequencies in a range from 10 MHz to 25 MHz.
13. The apparatus of any one of clauses 1-12, where the single array of M ultrasonic receiver elements is linearly arranged.
14. The apparatus of clause 1, where the N light sources include two or more vertical-cavity surface-emitting lasers (VCSELs).
15. An apparatus, including: a light source system configured to provide light to a target object on an outer surface of the apparatus, the light source system including N light sources; an ultrasonic receiver system configured to receive ultrasonic waves generated by the target object responsive to the light from the light source system, the ultrasonic receiver system including a single array of M ultrasonic receiver elements; and control means for: controlling the light source system to provide light to the target object; receiving ultrasonic receiver signals from each of a plurality of the M ultrasonic receiver elements in the single array, the ultrasonic receiver signals corresponding to the ultrasonic waves generated by the target object responsive to the light; detecting an artery within the target object based on the ultrasonic receiver signals; and estimating a blood pressure within the artery based on the ultrasonic receiver signals.
16. The apparatus of clause 15, where the apparatus is configured to be a hand-held device.
17. The apparatus of clause 15 or clause 16, where: the control means includes means for estimating a change in cross-sectional area of the artery based at least in part the ultrasonic receiver signals; and estimating the blood pressure is based in part on the change in the cross-sectional area of the artery.
18. A blood pressure estimation method, including: controlling, by a control system of an apparatus, a light source system of the apparatus to provide light to a target object on an outer surface of the apparatus; receiving, by the control system, ultrasonic receiver signals from each of a plurality of M ultrasonic receiver elements in a single array, the ultrasonic receiver signals corresponding to ultrasonic waves generated by the target object responsive to the light; detecting, by the control system, an artery within the target object based on the ultrasonic receiver signals; and estimating, by the control system, a blood pressure within the artery based on the ultrasonic receiver signals.
19. The method of clause 18, further including estimating a change in cross-sectional area of the artery based at least in part the ultrasonic receiver signals, where estimating the blood pressure is based in part on the change in the cross-sectional area of the artery.
20. The method of clause 19, further including estimating a change in a blood flow rate within the artery based on the ultrasonic receiver signals, where estimating the blood pressure is based in part on the change in the blood flow rate within the artery.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

1. An apparatus, comprising:
a light source system configured to provide light to a target object on an outer surface of the apparatus, the light source system including N light sources;
an ultrasonic receiver system configured to receive ultrasonic waves generated by the target object responsive to the light from the light source system, the ultrasonic receiver system including a single array of M ultrasonic receiver elements; and
a control system configured to:
control the light source system to provide light to the target object;
receive ultrasonic receiver signals from each of a plurality of the M ultrasonic receiver elements in the single array, the ultrasonic receiver signals corresponding to the ultrasonic waves generated by the target object responsive to the light;
detect an artery within the target object based on the ultrasonic receiver signals; and
estimate a blood pressure within the artery based on the ultrasonic receiver signals.

2. The apparatus of claim 1, wherein the apparatus is configured to be worn on a human wrist and wherein the artery is a radial artery.

3. The apparatus of claim 1, wherein the control system is further configured to apply a receiver-side beamforming process to the ultrasonic receiver signals, to produce a beamformed ultrasonic receiver image.

4. The apparatus of claim 3, wherein:
the control system is further configured to estimate a change in cross-sectional area of the artery based at least in part on the beamformed ultrasonic receiver image; and
estimating the blood pressure is based in part on the change in the cross-sectional area of the artery.

5. The apparatus of claim 4, wherein:
the control system is further configured to estimate a change in a blood flow rate within the artery based on the ultrasonic receiver signals; and
estimating the blood pressure is based in part on the change in the blood flow rate within the artery and the change in cross-sectional area of the artery.

6. The apparatus of claim 5, wherein estimating the blood pressure is based in part on a derivative of the blood flow rate (Q) within the artery with respect to the cross-sectional area (A) of the artery (dQ/dA).

7. The apparatus of claim 6, wherein the control system is configured to estimate a pulse wave velocity based on dQ/dA.

8. The apparatus of claim 5, wherein the control system is configured to estimate the change in the blood flow rate within the artery based at least in part on speckle decorrelation-based velocimetry imaging, a continuous wave photoacoustic doppler method, a structured-illumination photoacoustic doppler method, cross-correlation-based flow imaging in a time domain or cross-correlation-based flow imaging in a spatial domain.

9. The apparatus of claim 4, further comprising a magnetic sensor system, wherein:
the control system is further configured to estimate a change in a blood flow rate within the artery based on magnetic sensor signals from the magnetic sensor system; and
estimating the blood pressure is based in part on the change in the blood flow rate within the artery and the change in cross-sectional area of the artery.

10. The apparatus of claim 4, further comprising an optical sensor system, wherein:
the control system is further configured to estimate a change in a blood flow rate within the artery based on optical sensor signals from the optical sensor system; and
estimating the blood pressure is based in part on the change in the blood flow rate within the artery and the change in cross-sectional area of the artery.

11. The apparatus of claim 1, wherein the control system is configured to receive ultrasonic receiver signals from the ultrasonic receiver system at a frame rate in a range from 1 KHz to 3 KHz.

12. The apparatus of claim 1, wherein the ultrasonic receiver signals include frequencies in a range from 10 MHz to 25 MHz.

13. The apparatus of claim 1, wherein the single array of M ultrasonic receiver elements is linearly arranged.

14. The apparatus of claim 1, wherein the N light sources comprise two or more vertical-cavity surface-emitting lasers (VCSELs).

15. An apparatus, comprising:
a light source system configured to provide light to a target object on an outer surface of the apparatus, the light source system including N light sources;
an ultrasonic receiver system configured to receive ultrasonic waves generated by the target object responsive to the light from the light source system, the ultrasonic receiver system including a single array of M ultrasonic receiver elements; and
control means for:
controlling the light source system to provide light to the target object;
receiving ultrasonic receiver signals from each of a plurality of the M ultrasonic receiver elements in the single array, the ultrasonic receiver signals corresponding to the ultrasonic waves generated by the target object responsive to the light;
detecting an artery within the target object based on the ultrasonic receiver signals; and
estimating a blood pressure within the artery based on the ultrasonic receiver signals.

16. The apparatus of claim 15, wherein the apparatus is configured to be a hand-held device.

17. The apparatus of claim 15, wherein:
the control means comprises means for estimating a change in cross-sectional area of the artery based at least in part the ultrasonic receiver signals; and
estimating the blood pressure is based in part on the change in the cross-sectional area of the artery.

18. A blood pressure estimation method, comprising:
controlling, by a control system of an apparatus, a light source system of the apparatus to provide light to a target object on an outer surface of the apparatus;
receiving, by the control system, ultrasonic receiver signals from each of a plurality of M ultrasonic receiver elements in a single array, the ultrasonic receiver signals corresponding to ultrasonic waves generated by the target object responsive to the light;
detecting, by the control system, an artery within the target object based on the ultrasonic receiver signals; and
estimating, by the control system, a blood pressure within the artery based on the ultrasonic receiver signals.

19. The method of claim 18, further comprising estimating a change in cross-sectional area of the artery based at least in part the ultrasonic receiver signals, wherein estimating the blood pressure is based in part on the change in the cross-sectional area of the artery.

20. The method of claim 19, further comprising estimating a change in a blood flow rate within the artery based on the ultrasonic receiver signals, wherein estimating the blood pressure is based in part on the change in the blood flow rate within the artery.
